(19) United States
(12) Patent Application Publication
(10) Pub. No.: US 2016/0350476 A1
(43) Pub. Date: Dec. 1, 2016

(54) ANTIVIRAL METHODS AND COMPOSITIONS

(71) Applicant: Agenovir Corporation, South San Francisco, CA (US)

(72) Inventors: Stephen R. Quake, Stanford, CA (US); Jianbin Wang, South San Francisco, CA (US)

(21) Appl. No.: 15/167,091

(22) Filed: May 27, 2016

Related U.S. Application Data
(60) Provisional application No. 62/168,183, filed on May 29, 2015.

(51) Int. Cl. G06F 19/18; A61K 31/7088; A61K 38/46

(52) U.S. Cl. CPC G06F 19/18; A61K 38/465; A61K 31/7088; A61K 48/00

(57) ABSTRACT

The invention relates to systems and methods for removing viral genetic sequences from host genomes by using a computer system to read a nucleotide string next to a protospacer adjacent motif (PAM) in the viral sequence, determine that the host genome lacks any region that matches the nucleotide string according to a predetermined similarity criteria and is adjacent to the PAM, and provide a guide sequence at least partially complementary to the nucleotide string. Providing the guide sequence may include synthesizing a guide RNA that includes a portion that is complementary to the nucleotide string.

### CROSS-REFERENCE TO RELATED APPLICATION

This application claims priority and benefit of U.S. Provisional Patent Application No. 62/168,183, filed May 29, 2015, the contents of which are incorporated by reference.

### TECHNICAL FIELD

The invention generally relates to method for removing viral genetic sequences from host organism genomes.

### BACKGROUND

Some viral infections lie dormant in a subject for a long time in what is called viral latency. Latency is a period in the viral life cycle in which, after initial infection, viral proliferation ceases. However, the viral genome is not fully eradicated. As a result, the virus can reactivate, causing acute infection and producing large amounts of progeny without any new infection. While this can produce symptoms such as cold sores, more serious ramifications of a latent infection include the possibility of transforming a cell, leading to uncontrolled cell division. Such viruses potentially include the human immunodeficiency virus (HIV), the herpes virus family (herpesviridae)—which includes Chicken-pox, Epstein-Barr virus, and Herpes simplex viruses (HSV-1, HSV-2), and hepatitis.

Nucleases—enzymes that digest nucleic acids—have been used to eradicate HIV-1 or Epstein-Barr virus. See e.g., Hu et al., 2014, PNAS 111(31):11461-11466 or Wang & Quake, 2014, PNAS 111(36):13157-13162, respectively. However, no reported method is known of for removing viral sequences from host genomes for other viruses such as herpes simplex virus (HSV)-1, HSV-2, varicella zoster virus (VZV), cytomegalovirus (CMV), human herpesvirus (HHV)-6, HHV-7, Kaposi's sarcoma-associated herpesvirus (KSHV), JC virus, BK virus, parvovirus b19, adeno-associated virus (AAV), and adenovirus. Thus there are a number of viruses that continue to affect people by latent infection and for which no reported method of eradicating the latent viral genome is yet known.

### SUMMARY

The invention provides methods and systems for removing viral sequences from host genomes by applying a set of rules to the viral and host genome sequences to provide a composition that can be used to target the viral sequence for degredation without interfering with the wellness of the host genome. The provided composition can include a guide RNA (gRNA) having a sequence that hybridizes to a target within the viral sequence. The composition may further include a targeted nuclease such as the cas9 enzyme, or a vector encoding such a nuclease, which uses the gRNA to bind exclusively to the viral genome and make double stranded cuts, thereby removing the viral sequence from the host. The sequence for the gRNA, or the guide sequence, can be determined by examination of the viral sequence to find regions of about 20 nucleotides that are adjacent to a protospacer adjacent motif (PAM) and that do not also appear in the host genome adjacent to the protospacer motif. Systems of the invention can further apply rules to design a guide sequence that satisfies certain similarity criteria (e.g., at least 60% identical with identity biased toward regions closer to the PAM) so that a gRNA/cas9 complex made according to the guide sequence will bind to and digest specified features or targets in the viral sequence without interfering with the host genome. Since the system can use a viral sequence and reference to a host genome to provide a gRNA designed to target that virus against the background of that host, the system can be used to provide materials for the removal of a latent viral infection, even where no known reported methods have addressed that virus. Thus systems and methods of the invention provide a design and synthesis pipeline for high-performance gRNA/nuclease compositions to eliminate latent virus genomes without harming human genomic background. The design and synthesis pipelines are of general applicability and can be used to address virus not yet targeted for removal or even not yet fully known or understood.

In certain aspects, the invention provides a method for removing a viral sequence from a host genome. The method includes using a computer system comprising a processor coupled to memory to read a nucleotide string next to a protospacer adjacent motif (PAM) (e.g., NGG, where N is any nucleotide) in the viral sequence. The computer system determines that the host genome lacks any region that (1) matches the nucleotide string according to a predetermined similarity criteria and (2) is also adjacent to the PAM. The computer system provides a guide sequence at least partially complementary to the nucleotide string. Providing the guide sequence may include synthesizing a guide RNA that includes a portion that is complementary to the nucleotide string.

The predetermined similarity criteria can include, for example, a requirement of at least 12 matching nucleotides within 20 nucleotides 5′ to the PAM and may also include a requirement of at least 7 matching nucleotides within 10 nucleotides 5′ to the PAM. The method may include receiving annotations for the viral sequence, wherein the annotations identify features of the viral sequence and finding the nucleotide string next to a protospacer adjacent motif (PAM) in the viral sequence within a selected feature (e.g., a viral replication origin, a terminal repeat, a replication factor binding site, a promoter, a coding sequence, or a repetitive region) of the viral sequence. The viral sequence and the annotations may be obtained from a genome database. The method may be used to find more than one candidate target in a coding sequence of the viral sequence according to the reading and determining steps. The selection rules may favor the 5′-most candidate target as the guide sequence. A plurality of guide sequences according to the reading and determining steps may be provided. The method may preferentially select sequences with neutral (e.g., 40% to 60%) GC content.

In certain embodiments, the viral sequence is aligned to homologous sequences of related viral genomes to create a multiple sequence alignment and a conserved region is identified within the viral sequence (e.g., a region that spans a greater than average density of conserved positions within the multiple sequence alignment. The reading and determining steps may be performed within the conserved region to provide the guide sequence at least partially complementary to a portion of the conserved region.

In some embodiments, the method is used for finding more than one candidate target in the viral sequence and according to the reading and determining steps. In certain embodiments, the nucleotide string is validated in a validation assay prior to providing the guide sequence. The validation assay may include exposing the host genome and a nucleic acid having the viral sequence in vivo to an RNA at least partially complementary to the nucleotide string and a cas9 protein. Methods of the invention may include synthesizing an expression vector encoding the guide sequence (e.g., also including any combination of a cas9 gene, a viral replication origin, a promoter). Methods of the invention may be used to target a virus such as herpes simplex virus (HSV)-1, HSV-2, varicella zoster virus (VZV), cytomegalovirus (CMV), human herpesvirus (HHV)-6, HHV-7, Kaposi's sarcoma-associated herpesvirus (KSHV), JC virus, BK virus, parvovirus b19, adeno-associated virus (AAV), or adenovirus.

In related aspects, the invention provides a system for removing a viral sequence from a host genome. The system includes a computer system comprising processor coupled to memory and the system can be used for reading a nucleotide string next to a protospacer adjacent motif (PAM) in the viral sequence, determining that the host genome lacks any region that matches the nucleotide string according to a predetermined similarity criteria and is adjacent to the PAM, and providing a guide sequence at least partially complementary to the nucleotide string. Optionally, the system may be used for obtaining the viral sequence and the annotations from a genome database; synthesizing a guide RNA that includes a portion that is complementary to the nucleotide string; providing a plurality of guide sequences according to the reading and determining steps; or any combination thereof. The system may include an instrument for the synthesis of nucleic acids and the instrument may be operated to synthesize the guide RNA. The system may receive annotations for the viral sequence, wherein the annotations identify features of the viral sequence, and find the nucleotide string next to a protospacer adjacent motif (PAM) in the viral sequence within a selected feature of the viral sequence. The system may implement any of the specific methodologies described above. For example, the system may be operable to align the viral sequence to homologous sequences of related viral genomes to create a multiple sequence alignment, identify a conserved region within the viral sequence that spans a greater than average density of conserved positions within the multiple sequence alignment, and perform the reading and determining steps within the conserve region to provide the guide sequence at least partially complementary to a portion of the conserved region. The system may be used to synthesize an expression vector encoding the guide sequence and any of a cas9 gene, a viral replication origin, or a promoter. The system may be used to eliminate a latent infection of a virus such as herpes simplex virus (HSV)-1, HSV-2, varicella zoster virus (VZV), cytomegalovirus (CMV), human herpesvirus (HHV)-6, HHV-7, Kaposi's sarcoma-associated herpesvirus (KSHV), JC virus, BK virus, parvovirus b19, adeno-associated virus (AAV), and adenovirus.

### BRIEF DESCRIPTION OF THE DRAWINGS

FIG. 1 diagrams creating a gRNA to target viral genomic sequence.

FIG. 2 gives a diagram of a system according to embodiments of the invention.

FIG. 3 illustrates the use of method to synthesize a nucleic acid such as a gRNA.

FIG. 4 presents a user interface that may be provided to aid in target selection.

FIG. 5 describes an exemplary method for selecting a gRNA.

FIG. 6 outlines a similarity criteria according to certain embodiments.

FIG. 7 shows a multiple sequence alignment to identify conserved region.

FIG. 8 diagrams a vector according to certain embodiments.

FIG. 9 shows key parts in the HBV genome targeted by CRISPR guide RNAs.

FIG. 10 shows a gel resulting from an in vitro CRISPR assay against HBV.

### DETAILED DESCRIPTION

The invention relates to systems and methods for removing viral genetic sequences from host genomes by using a computer system to read a nucleotide string next to a protospacer adjacent motif (PAM) in the viral sequence, determine that the host genome lacks any region that matches the nucleotide string according to a predetermined similarity criteria and is adjacent to the PAM, and provide a guide sequence at least partially complementary to the nucleotide string. Providing the guide sequence may include synthesizing a guide RNA that includes a portion that is complementary to the nucleotide string.

Systems and methods of the invention may be used to provide one or more guide RNA (gRNA) for use by an RNA-guided endonuclease such as Cas9 to remove a viral sequence from a host genome. Cas9 (CRISPR associated protein 9) is an RNA-guided DNA endonuclease enzyme. Cas9 was found as part of the Streptococcus pyrogenes immune system, where it memorizes and later cuts foreign DNA by unwinding it to seek regions complementary to a 20 basepair spacer region of the guide RNA, where it then cuts. Cas9 can be used to make site-directed double strand breaks in DNA, which can lead to gene inactivation or the introduction of heterologous genes through non-homologous end joining and homologous recombination. Other exemplary tools for gene editing include zinc finger nucleases and TALEN proteins.

Cas9 can cleave nearly any sequence complementary to the guide RNA. Native Cas9 uses a guide RNA composed of two disparate RNAs that associate to make the guide—the CRISPR RNA (crRNA), and the trans-activating RNA (tracrRNA). Additionally or alternatively, Cas9 targeting may be simplified through the engineering of a chimeric single guide RNA (sgRNA).

Studies suggest that Cas9 contain RNase H and HNH endonuclease homologous domains which are responsible for cleavages of two target DNA strands, respectively. The sequence similar to RNase H has a RuvC fold (one member of RNase H family) and the HNH region folds as T4 Endo VII (one member of HNH endonuclease family). Previous works on Cas9 have demonstrated that HNH domain is responsible for complementary sequence cleavage of target DNA and RuvC is responsible for the non-complementary sequence.

CRISPR-based genome editing has been applied in human cells, and shown promise in curing genetic diseases (Cell Stem Cell. 2013, 13(6): 653-8). However, using targeted nuclease to address viruses has only been tried on a case-by-case basis. See e.g., Hu et al., 2014, PNAS 111(31):11461-11466 or Wang & Quake, 2014, PNAS 111(36):13157-13162. The invention provides systems and methods that can be used to design and evaluate antiviral gRNA/nuclease for use against a human background. The invention provides a pipeline for designing and producing high-performance antiviral guide RNA/nuclease to eliminate latent virus genomes without harming the human genomic background, as well as methods for creating antiviral compositions and systems that use one or more gRNA to target viral genomic sequence without affecting host genome sequence.

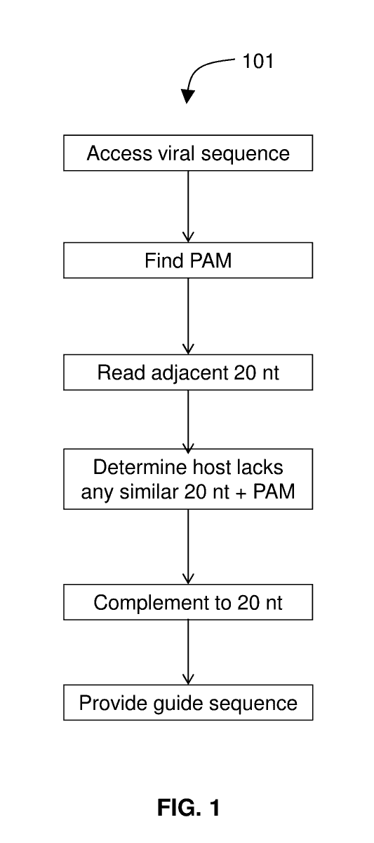

FIG. 1 diagrams a method 101 for creating a gRNA to target viral genomic sequence without affecting host genome sequence. The method includes using a computer system to access a viral genome and read a nucleotide string next to a protospacer adjacent motif (PAM) in the viral sequence. This may be done by scanning the viral genome to find a PAM. For cas9, the PAM is NGG, where N is any nucleotide. Additional background regarding the RNA-directed targeting by endonuclease is discussed in U.S. Pub. 2015/0050699; U.S. Pub. 20140356958; U.S. Pub. 2014/0349400; U.S. Pub. 2014/0342457; U.S. Pub. 2014/0295556; and U.S. Pub. 2014/0273037, the contents of each of which are incorporated by reference for all purposes. The computer scans through the viral sequence and finds an NGG. Upon finding NGG in the viral sequence, the computer reads the 20 nucleotides of the viral sequence that are adjacent to the NGG (i.e., the PAM). Those 20 nucleotides are provisionally considered as a potential sequence for the gRNA. To be used as the sequence for the gRNA, it is preferable to determine that the host genome lacks any region that (1) matches the nucleotide string according to some predetermined similarity criteria and (2) is also adjacent to a PAM within the host genome. Exemplary predetermined similarity criteria are discussed in greater detail but one straightforward similarity criteria is the requirement for a match. The computer scans the host genome to determine that the host genome lacks any such region (i.e., a 20 nucleotides with certain similarities to the sequence being provisionally considered and adjacent to a PAM). Once established that the host genome lacks such a region, the computer takes the complement of the sequence being provisional considered and provides it as a guide sequence—a sequence to be used in a gRNA. In certain embodiments, providing the guide sequence includes synthesizing a gRNA that includes a portion that is complementary to the nucleotide string. In some embodiments, methods and materials of the invention use a plasmid that includes a cas9 gene and at least one gene for a short guide RNA (sgRNA). The sgRNA is complementary to a portion of the viral genome.

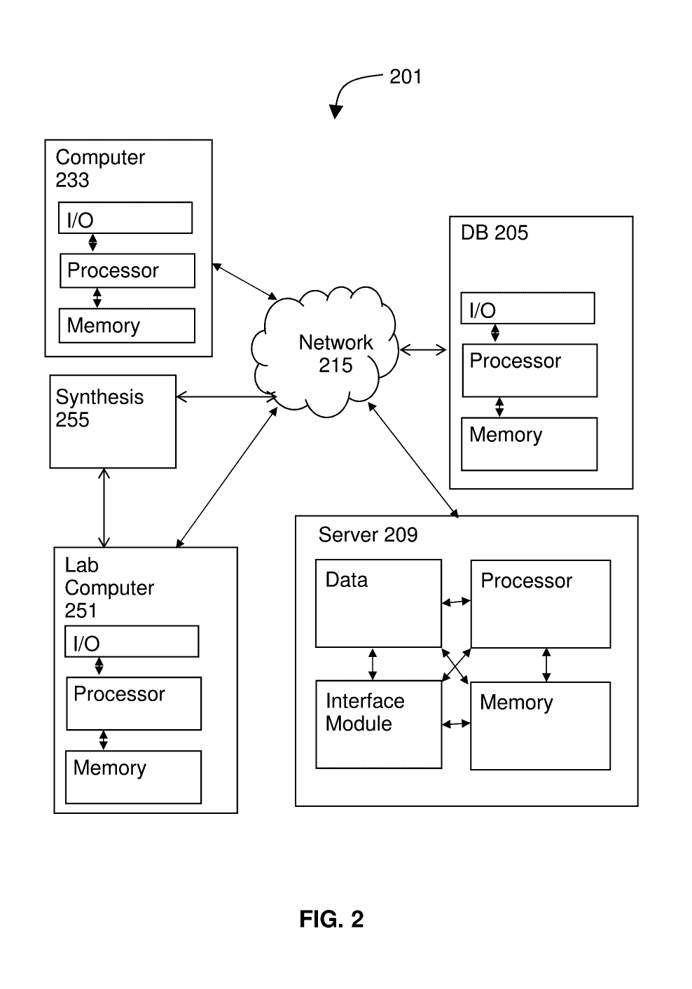

FIG. 2 gives a diagram of a system 201 according to embodiments of the invention. Preferably system 201 includes a computer 233 (e.g., laptop, desktop, or tablet) for use by a user and may also include a server computer 209. Server computer may have access to a database 205. System 201 may include a synthesis instrument 255 for creating gRNAs or other materials. The synthesis instrument 255 may optionally include or be operably coupled to its own, e.g., dedicated, analysis computer 251 (including an input/output mechanism, one or more processor, and memory). Additionally or alternatively, the instrument 255 may be operably coupled to the server 209 or the computer 233 via a communications network 215.

Each computer as illustrated in system 201 preferably includes a processor coupled to a memory and at least one input/output device.

Processor refers to any device or system of devices that performs processing operations. A processor will generally include a chip, such as a single core or multi-core chip, to provide a central processing unit (CPU). A process may be provided by a chip from Intel or AMD. A processor may be any suitable processor such as the microprocessor sold under the trademark XEON E7 by Intel (Santa Clara, Calif.) or the microprocessor sold under the trademark OPTERON 6200 by AMD (Sunnyvale, Calif.).

Memory refers a device or system of devices that store data or instructions in a machine-readable format. Memory may include one or more sets of instructions (e.g., software) which, when executed by one or more of the processors of the disclosed computers can accomplish some or all of the methods or functions described herein. Preferably, each computer includes a non-transitory memory such as a solid state drive, flash drive, disk drive, hard drive, subscriber identity module (SIM) card, secure digital card (SD card), micro SD card, or solid-state drive (SSD), optical and magnetic media, others, or a combination thereof.

An input/output device is a mechanism or system for transferring data into or out of a computer. Exemplary input/output devices include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

System 201 or components of system 201 may be used to perform methods described herein. Instructions for any method step may be stored in memory and a processor may execute those instructions. Any of the software can be physically located at various positions, including being distributed such that portions of the functions are implemented at different physical locations. System 201 or components of system 201 may be used in methods for removing a viral sequence from a host genome. Specifically, components illustrated in FIG. 2 may be operated to read a nucleotide string next to a protospacer adjacent motif (PAM) in a viral sequence, determine that the host genome lacks any region that matches the nucleotide string according to a predetermined similarity criteria and is adjacent to a host PAM, and provide a guide sequence at least partially complementary to the nucleotide string.

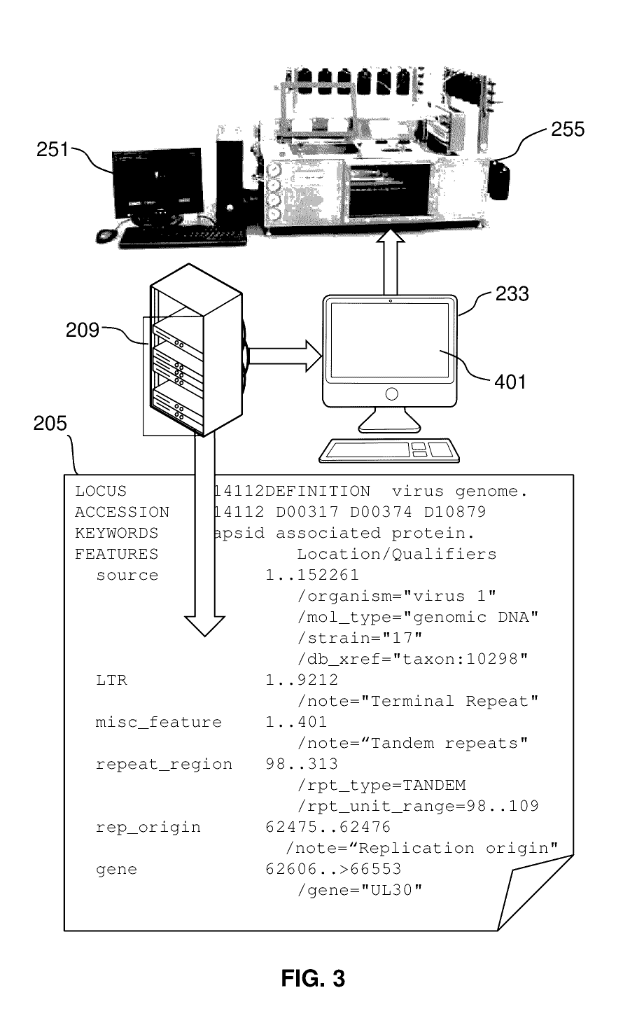

FIG. 3 illustrates the use of method 101 to synthesize a nucleic acid such as a gRNA, a vector such as a plasmid, a template (e.g., for amplification or incorporation into a vector), or any other nucleic acid suitable for use in the targeted removal of viral genetic sequence from a host genome. As shown in FIG. 3, server computer 209 may access a viral genome from a database 205 such as GenBank.

In the illustrated example, the server computer 209 is obtaining the viral genome sequence as well as annotations identifying features in the viral genome. In some embodiments, systems and methods of the invention target key features within a viral genome for endonuclease digestion. Discussed in greater detail below, this feature targeting can refer to features reported in annotations as found, for example, in the headers of files in GenBank format.

As shown in FIG. 3, computer 233 and server 209 are being used to design one or more gRNA. Following the steps of method 101 and applying the similarity criteria as well as other design parameters discussed below, the system 201, after reading a nucleotide string next to a protospacer adjacent motif (PAM) in the viral sequence, can then provide a guide sequence at least partially complementary to the nucleotide string. Note that computer 233 has a user-interface 401 by which a user can establish or select similarity criteria or other design parameters. Additionally, the guide sequence may be provided by display on user-interface 401. In certain embodiments, the guide sequence is provided by synthesizing a gRNA embodying the guide sequence. System 201 can synthesize the gRNA by operating synthesis instrument 255. A user may interact with instrument computer 251 to control operation of synthesis instrument 255.

Synthesis instrument 255 may be used to synthesize oligonucleotides such as gRNAs or single-guide RNAs (sgRNAs). Any suitable instrument or chemistry may be used to synthesize a gRNA. In some embodiments, the synthesis instrument 255 is the MerMade 4 DNA/RNA synthesizer from Bioautomation (Irving, Tex.). Such an instrument can synthesize up to 12 different oligonucleotides simultaneously using either 50, 200, or 1,000 nanomole prepacked columns. The synthesis instrument 255 can prepare a large number of molecules per run. These molecules (e.g., oligos) can be made using individual prepacked columns (e.g., arrayed in groups of 96) or well-plates.

By the described means, systems and methods of the invention may be used to provide gRNA for antiviral applications particularly against the background of a human genome (e.g., for eradicating viral genetic sequences from a human genome where there is a latent viral infection). In some embodiments, system 201 is operable to provide the synthetic nucleic acids that include the sequence of the gRNA—for example, either to provide the gRNAs themselves or to provide elements to be cloned or combined into vectors such as plasmids encoding the gRNA. An important feature of the invention is that system 201 may be used to design the gRNA. In fact, given sufficient inputs (e.g., the identity of a virus or genome accession number for a genome databank, the background or human genome sequence, and optionally annotations identifying features in the viral genetic sequence), system 201 may be operable to automatically design gRNAs and provide the sequence of a gRNA for use in antiviral applications.

The invention includes the creation of a set of rules that, taken together and embodied in the control systems 209/233, provide high-performance guide RNAs for eradicating latent viral infections, which rules and systems provide a tool for addressing viruses that have not yet been studied or addressed. That is, using systems of the invention, a virus that has not yet been addressed by a targeting endonuclease can have its genome digested out of a human genome. The system operates using the viral genome, the host genome, and preferably a set of annotations to aid in identifying targets. To obtain these ends, the system embodies the aforementioned set of rules to be used in automatically (by system 201) design high-performance antiviral guide RNA.

Any development environment or language known in the art may be used to implement embodiments of the invention. Exemplary languages, systems, and development environments include Perl, C++, Python, Ruby on Rails, JAVA, Groovy, Grails, Visual Basic .NET. An overview of resources useful in the invention is presented in Barnes (Ed.), Bioinformatics for Geneticists: A Bioinformatics Primer for the Analysis of Genetic Data, Wiley, Chichester, West Sussex, England (2007) and Dudley and Butte, A quick guide for developing effective bioinformatics programming skills, PLoS Comput Biol 5(12):e1000589 (2009).

In some embodiments, methods are implemented by a computer application developed in Perl (e.g., optionally using BioPerl). See Tisdall, Mastering Perl for Bioinformatics, O'Reilly & Associates, Inc., Sebastopol, Calif. 2003. In some embodiments, applications are developed using BioPerl, a collection of Perl modules that allows for object-oriented development of bioinformatics applications. BioPerl is available for download from the website of the Comprehensive Perl Archive Network (CPAN). See also Dwyer, Genomic Perl, Cambridge University Press (2003) and Zak, CGI/Perl, 1st Edition, Thomson Learning (2002).

In certain embodiments, applications are developed using Java and optionally the BioJava collection of objects, developed at EBI/Sanger in 1998 by Matthew Pocock and Thomas Down. BioJava provides an application programming interface (API) and is discussed in Holland, et al., BioJava: an open-source framework for bioinformatics, Bioinformatics 24(18):2096-2097 (2008). Programming in Java is discussed in Liang, Introduction to Java Programming, Comprehensive (8th Edition), Prentice Hall, Upper Saddle River, N.J. (2011) and in Poo, et al., Object-Oriented Programming and Java, Springer Singapore, Singapore, 322 p. (2008).

Applications can be developed using the Ruby programming language and optionally BioRuby, Ruby on Rails, or a combination thereof. Ruby or BioRuby can be implemented in Linux, Mac OS X, and Windows as well as, with JRuby, on the Java Virtual Machine, and supports object oriented development. See Metz, Practical Object-Oriented Design in Ruby: An Agile Primer, Addison-Wesley (2012) and Goto, et al., BioRuby: bioinformatics software for the Ruby programming language, Bioinformatics 26(20):2617-2619 (2010).

Systems and methods of the invention can be developed using the Groovy programming language and the web development framework Grails. Grails is an open source model-view-controller (MVC) web framework and development platform that provides domain classes that carry application data for display by the view. Grails provides a development platform for applications including web applications, as well as a database and an object relational mapping framework called Grails Object Relational Mapping (GORM). The GORM can map objects to relational databases and represent relationships between those objects. GORM relies on the Hibernate object-relational persistence framework to map complex domain classes to relational database tables. Grails further includes the Jetty web container and server and a web page layout framework (SiteMesh) to create web components. Groovy and Grails are discussed in Judd, et al., Beginning Groovy and Grails, Apress, Berkeley, Calif., 414 p. (2008); Brown, The Definitive Guide to Grails, Apress, Berkeley, Calif., 618 p. (2009).

Such tools can be used to control systems 209/233 to provide high-performance guide RNAs. Experience with designing guide RNA/nuclease for human genome engineering can serve as a primer for antiviral guide RNA/nuclease design. Due to the existence of human genomes background in the infected cells, a set of steps are provided to ensure high efficiency against the viral genome and low off-target effect on the human genome. Those steps may include (1) target selection within viral genome, (2) avoiding PAM+target sequence in host genome, (3) methodologically selecting viral target that is conserved across strains, (4) selecting target with appropriate GC content, (5) control of nuclease expression in cells, (6) vector design, (7) validation assay, others and various combinations thereof. Systems and methods of the invention may be implemented and controlled using software designed to implement those steps using system 201.

1. Target Selection within Viral Genome

One important difference between nuclease-based human genome editing and antiviral therapy relates to the objective. The purpose of human genome editing is to make controlled modifications at specific sites, while antiviral therapy according to the present invention aims for systematic destruction of the viral genome. Although guide RNA can target a wide selection of sequences within the viral genome, the resulting endonuclease digestion may lead to dramatically different physiological effect. Therefore, the selection of viral targets should be considered at a higher level, beyond a specific gene. To aid in the selection of viral targets, the invention provides tools that automatically determine or suggest certain targets based on certain rules, and can provide a menu of options for final selection by a user.

The system 201 operates to obtain a viral reference genome, preferably annotated, as illustrated in FIG. 3. This can be achieved by searching in NCBI and viral specific consortium database. The reference genome can serve as a design guide.

In certain embodiments, the system 201 references the annotations to select targets within certain categories such as (i) latency related targets, (ii) infection and symptom related targets, and (iii) structure related targets. The system 201 can read through the annotations (e.g., using pattern matching such as regular expressions, sometimes known as RegEx) and find the coordinates for key features (discussed in more detail below) such as terminal repeats, tandem repeats, or an origin of replication.

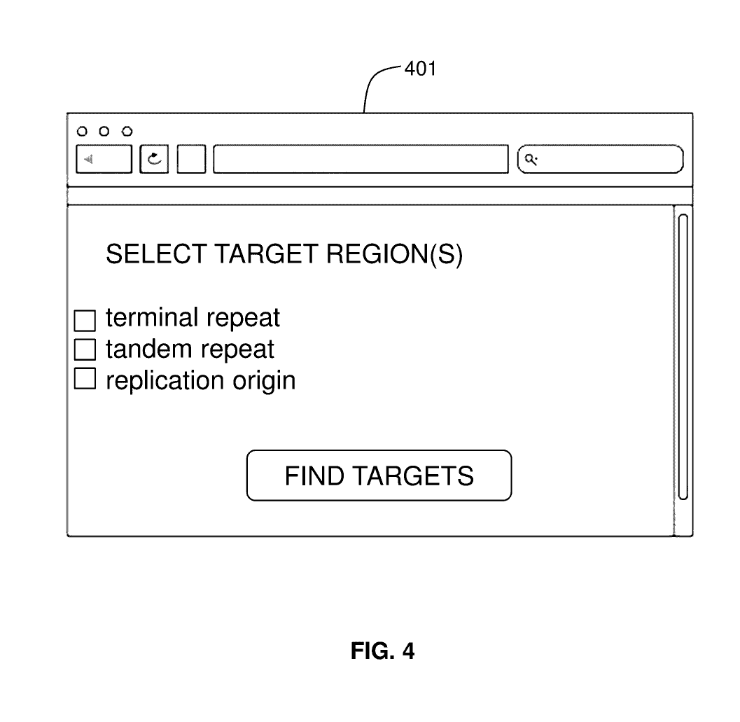

FIG. 4 presents a user interface 401 that may be provided by the system 201 to aid in target selection. In some embodiments, the system 201 provides a menu of pre-selected target options for final selection by a user. In certain embodiments, the system 201 simply selects the targets automatically based on an order of preference (e.g., origin of replication>promoter>capsid protein). The invention includes that insight that potential targets fall into certain categories that—due to their biological significance—make those categories of targets good candidates as targets for nuclease digestion.

A first category of targets for gRNA includes latency-related targets. The viral genome requires certain features in order to maintain the latency. These features include, but not limited to, master transcription regulators, latency-specific promoters, signaling proteins communicating with the host cells, etc. If the host cells are dividing during latency, the viral genome requires a replication system to maintain genome copy level. Viral replication origin, terminal repeats, and replication factors binding to the replication origin are great targets. Once the functions of these features are disrupted, the viruses may reactivate, which can be treated by conventional antiviral therapies.

A second category of targets for gRNA includes infection-related and symptom-related targets. Virus produces various molecules to facilitate infection. Once gained entrance to the host cells, the virus may start lytic cycle, which can cause cell death and tissue damage (HBV). In certain cases, such as HPV16, cell products (E6 and E7 proteins) can transform the host cells and cause cancers. Disrupting the key genome sequences (promoters, coding sequences, etc) producing these molecules can prevent further infection, and/or relieve symptoms, if not curing the disease.

A third category of targets for gRNA includes structure-related targets. Viral genome may contain repetitive regions to support genome integration, replication, or other functions. Targeting repetitive regions can break the viral genome into multiple pieces, which physically destroys the genome.

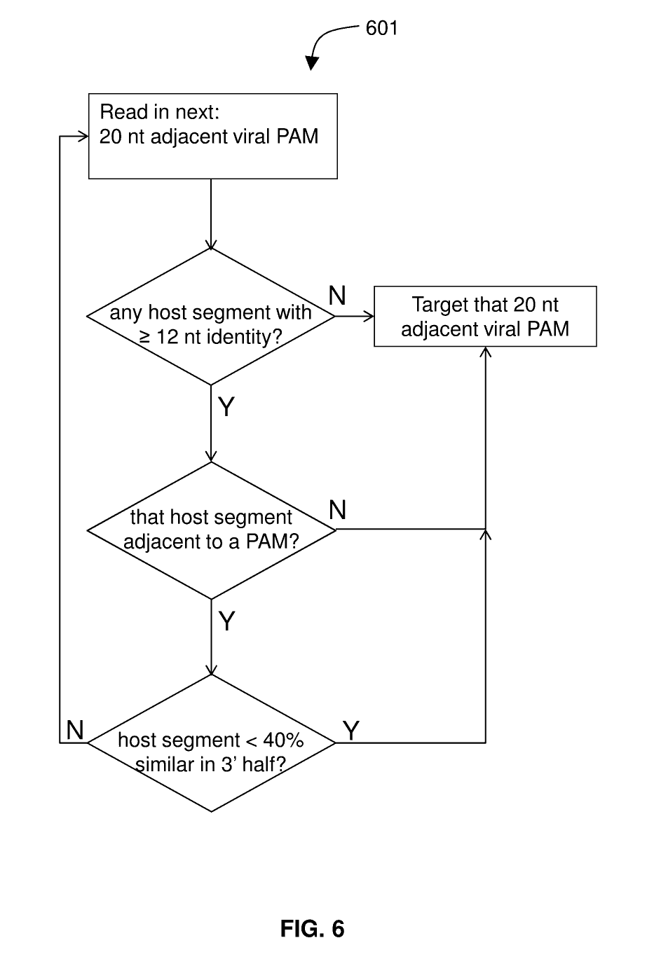

Design rules embodied in the disclosed design pipeline can include a rule preferring a 5′ bias in selection of targets. Specifically, where more than one candidate target is found in a coding sequence of the viral sequence according to the disclosed steps (e.g., FIG. 1 and/or FIG. 6), the system may automatically provide the 5′-most candidate target as the guide sequence.

When designing guide RNA against protein coding regions, it may be preferable to focus on the 5′ end, so that a single cutting could introduce insertion/deletion and frame shift early in the coding sequence. When combined with other guide RNAs, this design could potentially delete the majority of the gene body. For promoters and replication origins, one should identify the protein binding sites on DNA. Destruction of binding site by guide RNA/nuclease can abolish the binding affinity between DNA and proteins. As mentioned above, combination of multiple guide RNAs is essential for viral genome destruction. While the design of single RNA should maximize the sequence disruption effect, the placement of multiple guides also may be carefully considered, so that long stretch of essential sequences can be removed from the genome by the system 201. Furthermore, the resulting pieces of multiple nuclease digestion have a lower chance to be re-assembled back into a functional viral genome.

Once a broad targeting region or category is identified, the selection of specific guide RNAs may further involve reference to the following various steps or principles. For example, given a certain target region within a viral genetic sequence, system 201 may execute a structured set of rules to find a specific 20 nt target sequence within that target region.

### 2. Protospacer Adjacent Motif (PAM)

Each cas protein requires a specific PAM next to the targeted sequence (not in the guide RNA). This is the same as for human genome editing. The current understanding the guide RNA/nuclease complex binds to PAM first, then searches for homology between guide RNA and target genome. Sternberg et al., 2014, DNA interrogation by the CRISPR RNA-guided endonuclease Cas9, Nature 507(7490):62-67. Once recognized, the DNA is digested 3-nt upstream of PAM. These results suggest that off-target digestion requires PAM in the host DNA, as well as high affinity between guide RNA and host genome right before PAM.

Based on the aforementioned off-target digestion mechanism, the invention provides methods to avoid human genome digestion as follow. First, a candidate target gRNA in the viral genome must be selected.

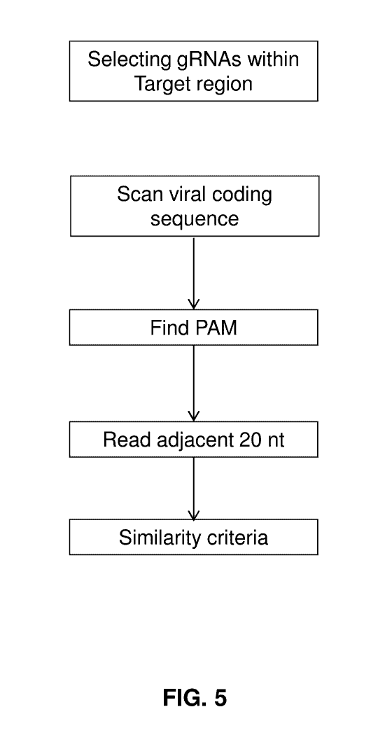

FIG. 5 describes an exemplary method for selecting a gRNA within the viral target region. The system 201 scans the viral coding sequence and finds the PAM for the nuclease that is to be used. For example, where the digestion system will include cas9, the system 201 scan the target for NGG, where N is any nucleotide. Upon finding the PAM in the viral genome, the system 201 reads the 20 nucleotide string adjacent to the PAM within the viral genome. This 20 nucleotide string is provisionally treated as a potential sequence for the gRNA. Finally selecting the nucleotide string for the gRNA involves determining if the nucleotide string satisfies a similarity criteria for any region within the host genome (i.e., a gRNA is only selected if there is no region within the host genome that is similar enough according to a defined criteria).

Any suitable similarity criteria may be used. For example, one similarity criteria may be the requirement of a perfect match for all 20 bases of the nucleotide string. Other criteria may include that 19 bases match, or 18, etc. In a preferred embodiment, the invention includes similarity criteria that balance the requirement of actually finding a useful gRNA with the probabilities of some matching portions in the host, i.e., the possibility that even without a perfect 20 nt match, some of the gRNA may still bind to the host genome and initiate nuclease action. The includes similarity criteria that minimize the off-target action against the host genome.

FIG. 6 outlines a similarity criteria 601 according to certain embodiments that can be automatically applied by system 201. To avoid digestion of host genome, the system applies a search criteria that embodies certain principles. The system 201 preferably tries to avoid any target sequence with any ≧12 nt DNA stretch homology to the human genome. When homology to human genome is inevitable, the guide RNA candidate not followed by PAM in the human genome would not lead to off-target digestion, and should be given priority. If homologous sequences and PAM both are present in the human genome, one should choose the guide RNA candidate with low homology (e.g., <40% similar) to human genome in the half next to PAM, where double strand break happens.

To reach these principles, as diagrammed in FIG. 6, the system 201 reads in a 20 nt nucleotide string adjacent a PAM in the viral sequence. The system 201 examines the host genome for any segment with ≧12 nt identity to the nucleotide string. If no such segment is found (N), then that nucleotide string is provided as the guide sequence to target that 20 nt in the viral genome. If such a segment is found in the human genome (Y), then the system 201 determines if that segment in the host genome is adjacent to a PAM. If that segment in the host genome is not adjacent to a PAM (N), then that nucleotide string is provided as the guide sequence to target that 20 nt in the viral genome. If that segment in the host genome is adjacent to a PAM (Y), then the system 201 determines if the half of that segment that is closest to the PAM is less than 40% similar to the nucleotide string. If the half of that segment that is closest to the PAM is less than 40% similar to the nucleotide string (Y), then that nucleotide string is provided as the guide sequence to target that 20 nt in the viral genome. If the half of that segment that is closest to the PAM is not less than 40% similar to the nucleotide string, then the system 201 reads in the next 20 nt nucleotide string in the viral genome sequence that is adjacent to a PAM and repeats the steps on that next candidate string. The cycle of steps is optionally repeated until at least one guide sequence is provided. Optionally, the steps may be repeated until several or all possible guide sequences are provided.

### 3. Conserved Viral Sequence

System 201 may be operated to automatically target portions of the viral genome that are highly conserved. Viral genomes are much more variable than human genomes. In order to target different strains, the guide RNA will preferably target conserved regions. As PAM is important to initial sequence recognition, it is also essential to have PAM in the conserved region. System 201 may be operated to locate instances of PAM in a conserved region. The system 201 may locate instances of PAM in a conserved region through the use of a multiple sequence alignment.

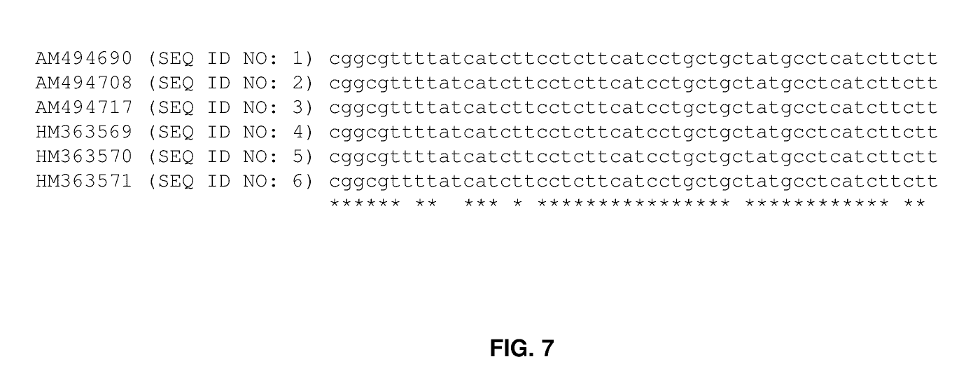

FIG. 7 shows a multiple sequence alignment that can be used to identify conserved region (here, HBV PreS1, conversed sites marked with *, noting that the multiple sequence alignment may contain many more than the 6 entries represented in FIG. 7).

Specifically, the system 201 may obtain a set of homologous sequences of related viral genomes and align the sequences to create a multiple sequence alignment, as shown in FIG. 7. In a multiple sequence alignment, each column represents in inference of homology at the represented site across the included sequences. A site may be said to be “conserved” if a substantial number (e.g., all) of the included sequences have the same nucleotide at that site. The presence of a conserved site in a multiple sequence alignment may be used as a justification for the inference that the site represents a conserved site in the viral genome. Using such a standard, the system 201 can identify conserved sites within a viral genome. System 201 may use the ability to identify conserved sites in a schema for identifying conserved regions. For example, a region in a genome that includes more than a certain density of conserved sites (e.g., more than the average density, or more than 50%) may be identified as a conserved region. By such means, the system 201 may identify a conserved region in the viral sequence (e.g., a region within the viral sequence that spans a greater than average density of conserved positions within the multiple sequence alignment. The system 201 may perform the reading and determining steps of method 101 within the conserved region and thereby provide a guide sequence that is at least partially complementary to a portion of the conserved region and thus targets a conserved region of the viral genome.

If no long stretch of conserved region is available, PAM and the region right before PAM should at least be conservative. This is based on the same principle mentioned in section 2, but in the opposite fashion here, to facilitate sequence recognition.

### 4. GC Content

High GC content improves stability between guide RNA and target genome, but also makes the target DNA difficult to be unwound. Therefore, guide RNA and the flanking target region should have medium GC content (40-60%), balancing the intra- and inter-target DNA stability. Once again, the region right before PAM should follow this GC content rule more strictly.

### 5. Control of Nuclease Expression in Cells

In a preferred embodiment, methods and systems of the invention are used to deliver a nucleic acid to cells. The nucleic acid delivered to the cells may include a gRNA having the determined guide sequence or the nucleic acid may include a vector, such as a plasmid, that encodes an enzyme that will act against the target genetic material. Expression of that enzyme allows it to degrade or otherwise interfere with the target genetic material. The enzyme may be a nuclease such as the Cas9 endonuclease and the nucleic acid may also encode one or more gRNA having the determined guide sequence.

The gRNA targets the nuclease to the target genetic material. Where the target genetic material includes the genome of a virus, gRNAs complementary to parts of that genome can guide the degredation of that genome by the nuclease, thereby preventing any further replication or even removing any intact viral genome from the cells entirely. By these means, latent viral infections can be targeted for eradication.

The host cells may grow at different rate, based on the specific cell type. High nuclease expression is necessary for fast replicating cells, whereas low expression help avoiding off-target cutting in non-infected cells. Control of nuclease expression can be achieved through several aspects. If the nuclease is expressed from a vector, having the viral replication origin in the vector can increase the vector copy number dramatically, only in the infected cells. Each promoter has different activities in different tissues. Gene transcription can be tuned by choosing different promoters. Transcript and protein stability can also be tuned by incorporating stabilizing or destabilizing (ubiquitin targeting sequence, etc) motif into the sequence.

The system 201 may provide specific promoters for the gRNA sequence, the nuclease (e.g., cas9), other elements, or combinations thereof. For example, in some embodiments, the gRNA is driven by a U6 promoter. A vector may be designed that includes a promoter for protein expression (e.g., using a promoter as described in the vector sold under the trademark PMAXCLONING by Lonza Group Ltd (Basel, Switzerland). Thus system 201 may provide an RNA polymerase promoter for the gRNA and a suitable promoter for proteins such as cas9. In some embodiments, system 201 is used to create a plasmid that includes some or all of those elements.

### 6. Vector Design

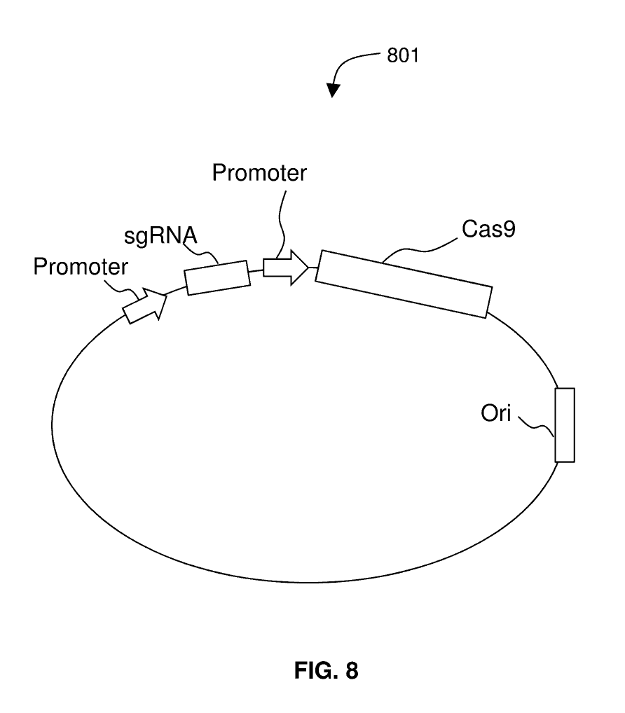

FIG. 8 diagrams a vector 801 according to certain embodiments. The vector 801 may be a plasmid (e.g., created by synthesis instrument 255 and recombinant DNA lab equipment). In certain embodiments, the plasmid includes a U6 promoter driven gRNA or chimeric guide RNA (sgRNA) and a ubiquitous promoter-driven cas9. Optionally, the vector 801 may include a marker such as EGFP fused after the cas9 protein to allow for later selection of cas9+ cells. It is recognized that cas9 can use a gRNA (similar to the CRISPR RNA (crRNA) of the original bacterial system) with a complementary trans-activating crRNA (tracrRNA) to target viral sequences complementary to the gRNA. It has also been shown that cas9 can be programmed with a single RNA molecule, a chimera of the gRNA and tracrRNA. The singe guide RNA (sgRNA) can be encoded in a plasmid and transcription of the sgRNA can provide the programming of cas9 and the function of the tracrRNA. See Jinek, 2012, A programmable dual-RNA-guided DNA endonuclease in adaptive bacterial immunity, Science 337:816-821 and especially FIG. 5A therein for background.

In one illustrative embodiment, systems and methods of the invention are employed to target latent infection of hepatitis B in a human host. Where the viral genome is a hepatitis B genome, the plasmid vector 801 may contain genes for one or more sgRNAs targeting locations in the hepatitis B genome such as PreS1, DR1, DR2, a reverse transcriptase (RT) domain of polymerase, an Hbx, and the core ORF. In a preferred embodiment, the one or more sgRNAs comprise one selected from the group consisting of sgHBV-Core and sgHBV-PreS1.

By delivering a vector 801 containing a provided guide sequence to human cells, transcription of the vector results in expression of the gRNA or sgRNA as well an mRNA that is transcribed to create cas9. The cas9 protein complexes with the gRNA and finds the target cutting site in the viral genetic sequence in the cells. For further illumination, the targeting mechanisms of cas9 are discussed in Sternberg, 2014, DNA interrogation by the CRISPR RNA-guided endonuclease Cas9, Nature 507(7490):62-67; Hsu, 2013, DNA targeting specificity of RNA-guided Cas9 nucleases, Nature Biotechnology 31(9):827-832; and Jinek, 2012, A programmable dual-RNA-guided DNA endonuclease in adaptive bacterial immunity, Science 337:816-821, the contents of each of which are incorporated by reference. Since the endonuclease is guided to the viral genetic sequence, it cleaves the sequence at the targeted locations. Since the targeted locations are selected to be within certain categories such as (i) latency related targets, (ii) infection and symptom related targets, or (iii) structure related targets, cleavage of those sequences inactivates the virus and removes it from the host. Since the targeting RNA (the gRNA or sgRNA) is designed to satisfy a similarity criteria 601 that matches the target in the viral genetic sequence without any off-target matching the host genome, the latent viral genetic material is removed from the host without any interference with the host genome. Thus systems and methods of the invention provide design and synthesis pipelines that can be used to eradicate latent viral infections and that may particularly be used to address viruses that have not yet been studied for eradication such as herpes simplex virus (HSV)-1, HSV-2, varicella zoster virus (VZV), cytomegalovirus (CMV), human herpesvirus (HHV)-6, HHV-7, Kaposi's sarcoma-associated herpesvirus (KSHV), JC virus, BK virus, parvovirus b19, adeno-associated virus (AAV), and adenovirus.

### 7. Validation Assay

It may preferable and useful to perform an in vitro validation assay. For each gRNA candidate, an in vitro validation assay should use PCR primers designed to amplify a region of about 300 to 1000 bp that flanks the presumptive gRNA target site. The expected cutting site should reside toward the center of the amplicon, so that endonuclease digestion of the amplicon will result in products having sizes suitably distinct from the amplicon to be obvious (e.g., when ran out on a gel). In vitro transcription may be used to produce guide RNA. Combine guide RNA, cas9 protein and PCR amplicon flanking each target to perform initial endonuclease assay. Activity is evaluated based on the percentage of target DNA amplicon being digested.

In some embodiments, a cellular validation assay is performed. To test nuclease activity within cells, search for cells carrying target virus. Sequence the flanking region of each target to verify target sequence diversity. One can also clone the flanking sequence of the viral target and delivery the DNA to cells to produce a transient cell model. Perform cellular endonuclease assay with cas protein (directly delivered or produced in the cells from expression vector), guide RNA (directly delivered or produced in the cells from expression vector), and target DNA (viral genome or cloned viral fragment).

After incubation in cells, harvest cells and extract genomic DNA. If the viral DNA double strand breaks are expected to be repaired, small insertion and deletions may present around the cutting sites. One can amplify the flanking region with PCR, re-anneal DNA molecules and perform mismatch recognition assay. If long deletions are expected, one can also design primers to amplify the specific DNA product by end joining outside deletions.

If viral DNA is short (a few thousand base pairs), the DNA may not be repaired after digestion. One can use quantitative PCR with primers flanking the double strand breaks to evaluate the digestion efficiency.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1

### Targeting Hepatitis B Virus (HBV)

Methods and materials of the present invention may be used to apply targeted endonuclease to specific genetic material such as a latent viral genome like the hepatitis B virus (HBV). The invention further provides for the efficient and safe delivery of nucleic acid (such as a DNA plasmid) into target cells (e.g., hepatocytes). In one embodiment, methods of the invention use hydrodynamic gene delivery to target HBV.

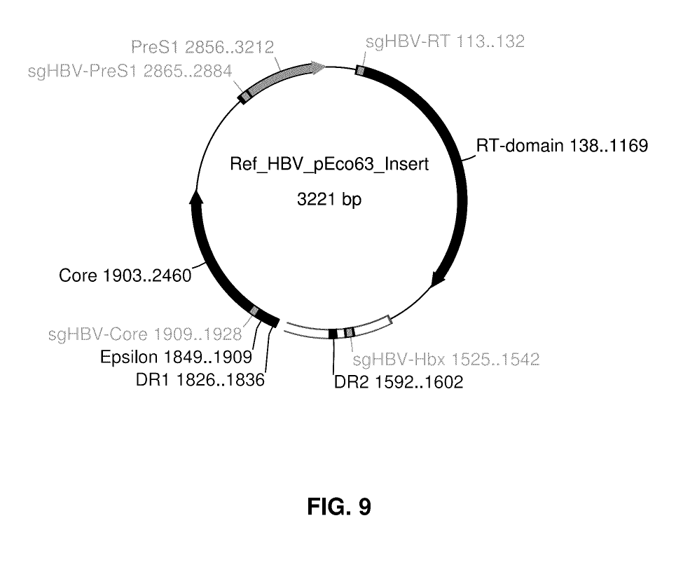

FIG. 9 diagrams the HBV genome. To remove the HBV genome from a human genome, a system 201 is used to read a nucleotide string next to a protospacer adjacent motif (PAM) in the HBV genome. It is determined that the human genome lacks any region that matches the nucleotide string according to a predetermined similarity criteria 601 and is adjacent to the PAM. That is, the system 201 scans through the HBV and finds an NGG (where N is any nucleotide). Upon finding NGG in the HBV genome, the system 201 reads the 20 nucleotides of the HBV genome adjacent the NGG (i.e., the PAM).

The system 201 then reads through the human genome and at any instance of NGG therein, the system 201 reads the 20 nt of the human genome adjacent that instance of the PAM (i.e., NGG). One of the processors in system 201 is used to compare that 20 of the human genome to the 20 nucleotides of the HBV genome.

Thus the system 201 searches the human genome for a feature of the form (“20 nucleotides of the HBV genome”+“NGG). If the system 201 identifies no such feature, then the 20 nucleotides are a candidate for targeting by enzymatic degredation.

It may be preferable to receive annotations for the HBV genome (i.e., that identify important features of the genome) and choose a candidate for targeting by enzymatic degredation that lies within one of those features, such as a viral replication origin, a terminal repeat, a replication factor binding site, a promoter, a coding sequence, and a repetitive region.

HBV, which is the prototype member of the family Hepadnaviridae, is a 42 nm partially double stranded DNA virus, composed of a 27 nm nucleocapsid core (HBcAg), surrounded by an outer lipoprotein coat (also called envelope) containing the surface antigen (HBsAg). The virus includes an enveloped virion containing 3 to 3.3 kb of relaxed circular, partially duplex DNA and virion-associated DNA-dependent polymerases that can repair the gap in the virion DNA template and has reverse transcriptase activities. HBV is a circular, partially double-stranded DNA virus of approximately 3200 bp with four overlapping ORFs encoding the polymerase (P), core (C), surface (S) and X proteins. In infection, viral nucleocapsids enter the cell and reach the nucleus, where the viral genome is delivered. In the nucleus, second-strand DNA synthesis is completed and the gaps in both strands are repaired to yield a covalently closed circular DNA molecule that serves as a template for transcription of four viral RNAs that are 3.5, 2.4, 2.1, and 0.7 kb long. These transcripts are polyadenylated and transported to the cytoplasm, where they are translated into the viral nucleocapsid and precore antigen (C, pre-C), polymerase (P), envelope L (large), M (medium), S (small)), and transcriptional transactivating proteins (X). The envelope proteins insert themselves as integral membrane proteins into the lipid membrane of the endoplasmic reticulum (ER). The 3.5 kb species, spanning the entire genome and termed pregenomic RNA (pgRNA), is packaged together with HBV polymerase and a protein kinase into core particles where it serves as a template for reverse transcription of negative-strand DNA. The RNA to DNA conversion takes place inside the particles.

Numbering of basepairs on the HBV genome is based on the cleavage site for the restriction enzyme EcoR1 or at homologous sites, if the EcoR1 site is absent. However, other methods of numbering are also used, based on the start codon of the core protein or on the first base of the RNA pregenome. Every base pair in the HBV genome is involved in encoding at least one of the HBV protein. However, the genome also contains genetic elements which regulate levels of transcription, determine the site of polyadenylation, and even mark a specific transcript for encapsidation into the nucleocapsid. The four ORFs lead to the transcription and translation of seven different HBV proteins through use of varying in-frame start codons. For example, the small hepatitis B surface protein is generated when a ribosome begins translation at the ATG at position 155 of the adw genome. The middle hepatitis B surface protein is generated when a ribosome begins at an upstream ATG at position 3211, resulting in the addition of 55 amino acids onto the 5′ end of the protein.

ORF P occupies the majority of the genome and encodes for the hepatitis B polymerase protein. ORF S encodes the three surface proteins. ORF C encodes both the hepatitis e and core protein. ORF X encodes the hepatitis B X protein. The HBV genome contains many important promoter and signal regions necessary for viral replication to occur. The four ORFs transcription are controlled by four promoter elements (preS1, preS2, core and X), and two enhancer elements (Enh I and Enh II). All HBV transcripts share a common adenylation signal located in the region spanning 1916-1921 in the genome. Resulting transcripts range from 3.5 nucleotides to 0.9 nucleotides in length. Due to the location of the core/pregenomic promoter, the polyadenylation site is differentially utilized. The polyadenylation site is a hexanucleotide sequence (TATAAA) as opposed to the canonical eukaryotic polyadenylation signal sequence (AATAAA). The TATAAA is known to work inefficiently (9), suitable for differential use by HBV.

There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in-frame start (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. The function of the protein coded for by gene X is not fully understood but it is associated with the development of liver cancer. It stimulates genes that promote cell growth and inactivates growth regulating molecules.

With reference to FIG. 9, HBV starts its infection cycle by binding to the host cells with PreS1. Guide RNA against PreS1 locates at the 5′ end of the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation. Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces.

HBV replicates its genome by reverse transcription of an RNA intermediate. The RNA templates is first converted into single-stranded DNA species (minus-strand DNA), which is subsequently used as templates for plus-strand DNA synthesis. DNA synthesis in HBV use RNA primers for plus-strand DNA synthesis, which predominantly initiate at internal locations on the single-stranded DNA. The primer is generated via an RNase H cleavage that is a sequence independent measurement from the 5′ end of the RNA template. This 18 nt RNA primer is annealed to the 3′ end of the minus-strand DNA with the 3′ end of the primer located within the 12 nt direct repeat, DR1. The majority of plus-strand DNA synthesis initiates from the 12 nt direct repeat, DR2, located near the other end of the minus-strand DNA as a result of primer translocation. The site of plus-strand priming has consequences. In situ priming results in a duplex linear (DL) DNA genome, whereas priming from DR2 can lead to the synthesis of a relaxed circular (RC) DNA genome following completion of a second template switch termed circularization. It remains unclear why hepadnaviruses have this added complexity for priming plus-strand DNA synthesis, but the mechanism of primer translocation is a potential therapeutic target. As viral replication is necessary for maintenance of the hepadnavirus (including the human pathogen, hepatitis B virus) chronic carrier state, understanding replication and uncovering therapeutic targets is critical for limiting disease in carriers.

In some embodiments, systems and methods of the invention target the HBV genome by finding a nucleotide string within a feature such as PreS1. Guide RNA against PreS1 locates at the 5′ end of the coding sequence. Thus it is a good candidate for targeting because it represents one of the 5′-most targets in the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation. Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces. In some embodiments, method of the invention include creating one or several guide RNAs against key features within a genome such as the HBV genome shown in FIG. 9.

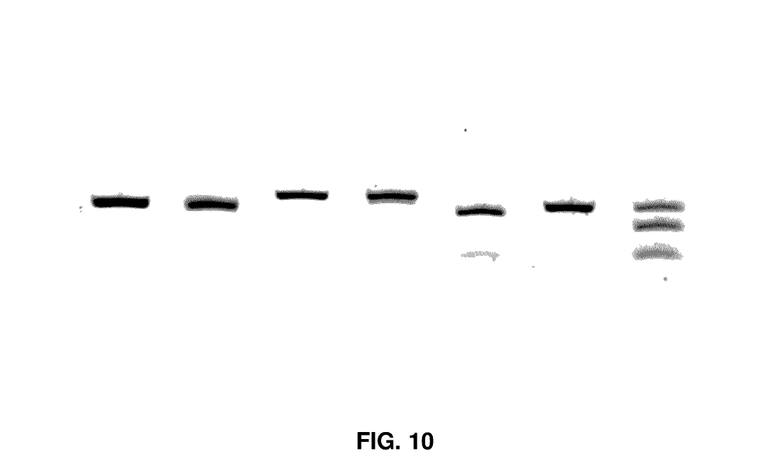

FIG. 9 shows key parts in the HBV genome targeted by CRISPR guide RNAs. To achieve the CRISPR activity in cells, expression plasmids coding cas9 and guide RNAs are delivered to cells of interest (e.g., cells carrying HBV DNA). To demonstrate in an in vitro assay, anti-HBV effect may be evaluated by monitoring cell proliferation, growth, and morphology as well as analyzing DNA integrity and HBV DNA load in the cells. The described method may be validated using an in vitro assay. To demonstrate, an in vitro assay is performed with cas9 protein and DNA amplicons flanking the target regions. Here, the target is amplified and the amplicons are incubated with cas9 and a gRNA having the selected nucleotide sequence for targeting. As shown in FIG. 10, DNA electrophoresis shows strong digestion at the target sites.

FIG. 10 shows a gel resulting from an in vitro CRISPR assay against HBV. Lanes 1, 3, and 6: PCR amplicons of HBV genome flanking RT, Hbx-Core, and PreS1. Lane 2, 4, 5, and 7: PCR amplicons treated with sgHBV-RT, sgHBV-Hbx, sgHBV-Core, sgHBV-PreS1. The presence of multiple fragments especially visible in lanes 5 and 7 show that sgHBV-Core and sgHBV-PreS1 provide especially attractive targets in the context of HBV and that use of systems and methods of the invention may be shown to be effective by an in vitro validation assay.

What is claimed is:

1. A method for removing a viral sequence from a host genome, the method comprising using a computer system comprising processor coupled to memory for:
reading a nucleotide string next to a protospacer adjacent motif (PAM) in the viral sequence;
determining that the host genome lacks any region that matches the nucleotide string according to a predetermined similarity criteria and is adjacent to the PAM; and
providing a guide sequence at least partially complementary to the nucleotide string.

2. The method of claim 1, wherein providing the guide sequence comprises synthesizing a guide RNA that includes a portion that is complementary to the nucleotide string.

3. The method of claim 1, wherein the PAM is NGG, wherein N is any nucleotide.

4. The method of claim 1, wherein the host genome is a human genome.

5. The method of claim 1, wherein the predetermined similarity criteria requires at least 12 matching nucleotides within 20 nucleotides 5′ to the PAM.

6. The method of claim 5, wherein the predetermined similarity criteria further requires at least 7 matching nucleotides within 10 nucleotides 5′ to the PAM.

7. The method of claim 1, further comprising:
receiving annotations for the viral sequence, wherein the annotations identify features of the viral sequence; and
finding the nucleotide string next to a protospacer adjacent motif (PAM) in the viral sequence within a selected feature of the viral sequence.

8. The method of claim 7, further comprising:
obtaining the viral sequence and the annotations from a genome database.

9. The method of claim 7, wherein the selected feature comprises one selected from the group consisting of: a viral replication origin, a terminal repeat, a replication factor binding site, a promoter, a coding sequence, and a repetitive region.

10. The method of claim 1, further comprising:
finding more than one candidate target in a coding sequence of the viral sequence according to the reading and determining steps; and
providing the 5′-most candidate target as the guide sequence.

11. The method of claim 1, further comprising providing a plurality of guide sequences according to the reading and determining steps.

12. The method of claim 1, further comprising:
aligning the viral sequence to homologous sequences of related viral genomes to create a multiple sequence alignment;
identifying a conserved region within the viral sequence that spans a greater than average density of conserved positions within the multiple sequence alignment; and
performing the reading and determining steps within the conserve region to provide the guide sequence at least partially complementary to a portion of the conserved region.

13. The method of claim 1, further comprising:
finding more than one candidate target in the viral sequence and according to the reading and determining steps; and
preferentially selecting a guide sequence with a medium GC content.

14. The method of claim 1, further comprising validating the nucleotide string in a validation assay prior to providing the guide sequence.

15. The method of claim 1, wherein the validation assay comprises exposing the host genome and a nucleic acid having the viral sequence in vivo to an RNA at least partially complementary to the nucleotide string and a cas9 protein.

16. The method of claim 1, further comprising synthesizing an expression vector encoding the guide sequence.

17. The method of claim 16, wherein the expression vector further comprises a cas9 gene.

18. The method of claim 17, wherein the expression vector further comprises a viral replication origin.

19. The method of claim 18, wherein the expression vector further comprises a promoter.

20. The method of claim 1, wherein the viral sequence is from a virus selected from the group consisting of herpes simplex virus (HSV)-1, HSV-2, varicella zoster virus (VZV), cytomegalovirus (CMV), human herpesvirus (HHV)-6, HHV-7, Kaposi's sarcoma-associated herpesvirus (KSHV), JC virus, BK virus, parvovirus b19, adeno-associated virus (AAV), and adenovirus.


SEQUENCE LISTING (6 sequences)

SEQ ID NO: 1 - DNA, 50 bp, Hepatitis B virus
cggcgttttatcatcttcctcttcatcctgctgctatgcctcatcttctt

SEQ ID NO: 2 - DNA, 50 bp, Hepatitis B virus; identical to SEQ ID NO: 1

SEQ ID NO: 3 - DNA, 50 bp, Hepatitis B virus; identical to SEQ ID NO: 1

SEQ ID NO: 4 - DNA, 50 bp, Hepatitis B virus; identical to SEQ ID NO: 1

SEQ ID NO: 5 - DNA, 50 bp, Hepatitis B virus; identical to SEQ ID NO: 1

SEQ ID NO: 6 - DNA, 50 bp, Hepatitis B virus; identical to SEQ ID NO: 1